(19) United States
(12) Patent Application Publication
(10) Pub. No.: US 2005/0153999 A1
(43) Pub. Date: Jul. 14, 2005

(54) Pharmaceutical compositions

(75) Inventor: Mengwei Hu, Twp. Of Washington, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 11/017,156

(22) Filed: Dec. 20, 2004

Related U.S. Application Data
(60) Provisional application No. 60/531,735, filed on Dec. 22, 2003.

(51) Int. Cl. A61K 31/4747

(52) U.S. Cl. 514/278

(57) ABSTRACT

Disclosed are pharmaceutical compositions comprising NK₁ Antagonists.

### CROSS REFERENCE TO RELATED APPLICATION

This application claims benefit of priority from U.S. Provisional patent application Ser. No. 60/531,735, filed Dec. 22, 2003 which is hereby incorporated by reference in it's entirety.

### BACKGROUND OF THE INVENTION

The invention relates to formulations containing an antagonist of the neuropeptide neurokinin-1 (NK₁ or NK-1) receptor and formulations containing the same.

Tachykinins are peptide ligands for neurokinin receptors. Neurokinin receptors, such as NK₁, NK₂ and NK₃, are involved in a variety of biological processes. They can be found in a mammal's nervous and circulatory systems, as well as in peripheral tissues. Consequently, the modulation of these types of receptors have been studied to potentially treat or prevent various mammalian disease states. For instance, NK₁ receptors have been reported to be involved in microvascular leakage and mucus secretion. Representative types of neurokinin receptor antagonists and their uses can be found in: U.S. Pat. No. 5,760,018 (1998) (pain, inflammation, migraine and emesis), U.S. Pat. No. 5,620,989 (1997) (pain, nociception and inflammation), WO 95/19344 (1995) (same), WO 94/13639 (1994) (same) and WO 94/10165 (1994) (same). Further types of NK₁ receptor antagonists can be found in Wu et al, Tetrahedron 56, 3043-3051 (2000); Rombouts et al, Tetrahedron Letters 42, 7397-7399 (2001); and Rogiers et al, Tetrahedron 57, 8971-8981 (2001).

Certain agents useful in treating such disorders must be able to administered to a patient. The aqueous solubility of drug substances plays an important role in the formulation of drug dosage forms. For the oral route of administration it is well experienced that, unless the substance has an aqueous solubility above 10 mg/ml over the pH-range 1-7, then potential absorption problems may occur. A solubility less than 1 mg/ml is likely to give dissolution-rate limited absorption because solubility and dissolution rate are interrelated.

Many important drugs have limited solubility in water, especially hydrophobic drugs. In order to attain the full expected therapeutic effect of such drugs, it is usually required that a solubilized form of the drug be administered to a patient.

A number of methods for solubilizing drugs have been developed that are based on the use of solvents or cosolvents, surfactants, complexation agents (e.g., cyclodextrins,), or complex drug carriers (e.g., liposomes). Each of the above methods has one or more drawbacks. Conventional surfactants and complexing agents have drawbacks of toxicity, and rapid precipitation of the solubilized drugs once administered to the patient or when otherwise diluted in an aqueous environment. Solvents and cosolvents can be toxic and irritating when injected into humans, such that the use of this solubilization approach is largely restricted to therapies for acute, life threatening diseases where medical experts are constantly in attendance to administer palliative treatments to counteract the adverse effects of the solvents/cosolvents. Water miscible solvents/cosolvents also possess the undesirable feature of allowing the drug to rapidly precipitate when an aqueous environment is contacted. Complex drug carriers, such as liposomes have limited utility due to the unstable nature of the carrier particles and the preferential uptake and localization of liposomal drugs to the reticuloendothelial system, namely, the liver and spleen. Accordingly, there exists a need for formulations that do not suffer from the above mentioned infirmities.

It would be beneficial to provide a formulation containing an NK, that has poor solubility that has improved physical and chemical stability. It would further be beneficial to provide a NK, antagonist that is effective for treating a variety of physiological disorders, symptoms and diseases while minimizing side effects. The invention seeks to provide these and other benefits, which will become apparent as the description progresses.

### SUMMARY OF THE INVENTION

Accordingly, there is disclosed a pharmaceutical composition comprising a compound having the chemical structure: 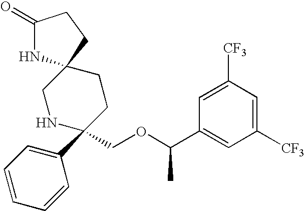
or a pharmaceutically acceptable salt thereof in admixture with a polyanionic β-cyclodextrin derivative with about one to about seven sodium sulfonate groups separated from the lipophilic cavity by at least one butyl ether spacer group.

There is also disclosed a pharmaceutically acceptable composition comprising a compound having the Formula (I): 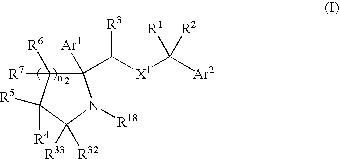
or a pharmaceutically-acceptable salt thereof, wherein
Ar¹ and Ar² are each independently selected from the group consisting of R¹⁷-heteroaryl and 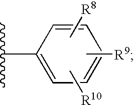
X¹ is —O—, —S—, —SO—, —SO₂—, —NR³⁴—, —N(COR¹²)— or —N(SO₂R¹⁵)—;
when X¹ is —SO—, —SO₂—, —N(COR¹²)— or —N(SO₂R¹⁵)—, then:
   R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or
when X¹ is —O—, —S— or —NR³⁴—, then:
   R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or R¹ and R², together with the carbon atom to which they are both attached, form a C═O group;
R³ is selected from the group consisting of H, C₁-C₆ alkyl, hydroxy(C₁-C₃ alkyl), C₃-C₈ cycloalkyl, —CH₂F, —CHF₂ and —CF₃;
each R⁶ is independently selected from the group consisting of H, C₁-C₆ alkyl and —OH;
each R⁷ is independently selected from the group consisting of H and C₁-C₆ alkyl;
n₂ is 1 to 4;
R⁴ and R⁵ are each independently selected from the group consisting of —(CR²⁸R²⁹)ₙ₁-G,
where,
   n₁ is 0 to 5; and
   G is H, —CF₃, —CHF₂, —CH₂F, —OH, —O—(C₁-C₆ alkyl), —OCH₂F, —OCHF₂, —OCF₃, —OCH₂CF₃, —O—(C₃-C₈ cycloalkyl), —O—(C₁-C₆)alkyl (C₃-C₈ cycloalkyl), —NR¹³R¹⁴, —SO₂NR¹³R¹⁴, —NR¹²SO₂R¹³, —NR¹²C(O)R¹⁴, —NR¹²C(O)OR¹³, —NR¹²(C(O)NR¹³ R¹⁴), —C(O)NR¹³R¹⁴, —C(O)OR¹³, —C₃-C₈ cycloalkyl, (R¹⁹)ᵣ-aryl, (R¹⁹)ᵣ-heteroaryl, —OC(O)R¹⁴, —OC(O)NR¹³R¹⁴, —C(═NOR¹⁴)(R¹³), —C(O)R¹³, —C(OR¹²)(R¹³)(R¹⁴), heterocycloalkenyl optionally substituted by 1 to 4 substituents independently selected from the group consisting of R³⁰ and R³¹, 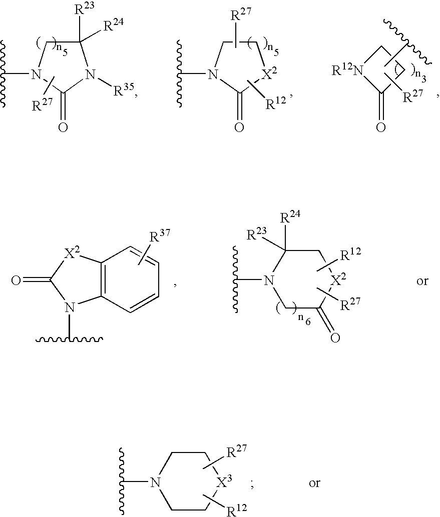
   R⁴ and R⁵ together are ═O, ═NOR¹²; or
R⁴ and R⁵, together with the carbon atom to which they are both attached, form a 4-to 8-membered heterocycloalkyl or heterocycloalkenyl ring containing 1 to 3 groups independently selected from X², provided that at least one X² is —NR³⁵—, —O—, —S—, —S(O)— or —SO₂—, the ring being optionally substituted with from 1 to 6 substituents independently selected from the group consisting of R³⁰ and R³¹;
provided that R⁴ and R⁵ are not both selected from the group consisting of H, alkyl and cycloalkyl;
further provided that, when one of R⁴ and R⁵ is —OH, then the other one of R⁴ and R⁵ is not alkyl or (R¹⁹)ᵣ-aryl;
R⁸, R⁹ and R¹⁰ are each independently selected from the group consisting of H, C₁-C₆ alkyl, C₃-C₈ cycloalkyl, —OR¹², halogen, —CN, —NO₂, —CF₃, —CHF₂, —CH₂F, —CH₂CF₃, —OCF₃, —OCHF₂, —OCH₂F, —OCH₂CF₃, —COOR², —CONR²¹R²², —OC(O)NR²¹ R²², —OC(O)R², —NR²¹ COR², —NR²¹ CO₂R⁵, —NR² CONR²¹R²², —NR² SO₂R⁵, —NR²¹R²², —SO₂NR², R²², —S(O)ₙ₆R¹⁵, (R¹⁹)ᵣ-aryl and (R¹⁹)ᵣ-heteroaryl;
R¹² is H, C₁-C₆ alkyl or C₃-C₈ cycloalkyl;
R¹³ and R¹⁴ are each independently selected from the group consisting of H, C₁-C₆ alkyl, C₃-C₈ cycloalkyl, (C₃-C₈)cycloalkyl(C₁-C₆)alkyl, —CH₂CF₃, aryl and heteroaryl; or
R¹³ and R¹⁴, together with the nitrogen atom to which they are both attached, form a 4- to 7-membered saturated or unsaturated ring that is optionally substituted with —OR², where one of the carbon atoms in the ring is optionally replaced by a heteroatom selected from the group consisting of —O—, —S— and —NR³⁴—;
n₆ is 0, 1 or 2;
R¹⁵ is C₁-C₆ alkyl, C₃-C₈ cycloalkyl, —CF₃ or —CH₂CF₃;
R¹⁸ is H, C₁-C₆ alkyl, C₃-C₈ cycloalkyl, (C₃-C₈)cycloalkyl(C₁-C₆)alkyl, hydroxy(C₂-C₆)alkyl or —P(O)(OH)₂;
each R¹⁹ is a substituent on the aryl or heteroaryl ring to which it is attached, and is independently selected from the group consisting of H, C₁-C₆ alkyl, C₃-C₈ cycloalkyl, C₁-C₆ alkoxy, —OH, halogen, —CN, —NO₂, —CF₃, —CHF₂, —CH₂F, —OCF₃, —OCHF₂, —OCH₂F, —O—(C₁-C₆ alkyl), —O—(C₃-C₈ cycloalkyl), —COOR¹², —CONR²¹R²², —OC(O)NR²¹R²²—OC(O)R², —NR²¹R²², —NR²¹COR¹², —NR²¹CO₂R¹², —NR²¹CONR²¹R²², —NR²¹SO₂R¹⁵ and —S(O)ₙ₆R¹⁵;
R²¹ and R²² are each independently selected from the group consisting of H, C₁-C₆ alkyl, C₃-C₈ cycloalkyl and benzyl; or
R²¹ and R²², together with the nitrogen atom to which they are both attached, form a 4- to 7-membered saturated or unsaturated ring, where one of the carbon atoms in the ring is optionally replaced by a heteroatom selected from the group consisting of —O—, —S— and —NR³⁴—;
R²³ and R²⁴ are each independently selected from the group consisting of H and C₁-C₆ alkyl; or
R²³ and R²⁴, together with the carbon atom to which they are both attached, form a C═O or cyclopropyl group;
R²⁷ is H, —OH or C₁-C₆ alkyl;
R²⁸ and R²⁹ are each independently selected from the group consisting of H and C₁-C₂ alkyl;
R³⁰ and R³¹ are each independently selected from the group consisting of H, —OH, C₁-C₆ alkyl, C₃-C₈ cycloalkyl, (C₃-C₈)cycloalkyl(C₁-C₆)alkyl and —C(O)NR¹³R¹⁴; or
R³⁰ and R³, together with the carbon atom to which they are both attached, form ═O, ═S, a cyclopropyl ring or ═NR³⁶;
R³² and R³³ are each independently selected from the group consisting of H and C₁-C₆ alkyl;
R³⁴ is H, C₁-C₆ alkyl, C₃-C₈ cycloalkyl, (C₃-C₈)cycloalkyl(C₁-C₆)alkyl or hydroxy(C₂-C₆)alkyl;
R³⁵ is H, C₁-C₆ alkyl, C₃-C₈ cycloalkyl, (C₃-C₈)cycloalkyl(C₁-C₆)alkyl, —P(O)(OH)₂, allyl, hydroxy(C₂-C₆)alkyl, (C₁-C₆)alkoxy(C₁-C₆)alkyl, —SO₂R¹⁵ or —(CH₂)₂—N(R¹²)—SO₂—R¹⁵;
R³⁶ is H, C₁-C₆ alkyl, C₃-C₈ cycloalkyl, (C₃-C₈)cycloalkyl(C₁-C₆)alkyl, —NO₂, —CN or OR¹²;
R³⁷ is 1 to 3 substituents independently selected from the group consisting of H, C₁-C₆ alkyl, —OH, C₁-C₆ alkoxy and halogen;
r is 1 to 3;
X² is —NR³⁵—, —O—, —S—, —S(O)—, —SO₂—, —CH₂—, —CF₂— or —CR¹²F—;
X³ is —NR³⁴—, —N(CONR¹³R¹⁴)—, —N(CO₂R¹³)—, —N(SO₂R¹⁵)—, —N(COR¹²)—, —N(SO₂NHR¹³)—, —O—, —S—, —S(O)—, —SO₂—, —CH₂—, —CF₂— or —CR¹²F—;
n₃ is 1 to 5; and
n₅ is 1 to 3;
   or a diastereomer, enantiomer, stereoisomer, regiostereomer, rotomer, tautomer prodrug or a pharmaceutically acceptable salt thereof in admixture with a polyanionic β-cyclodextrin derivative with about one to about seven sodium sulfonate groups separated from the lipophilic cavity by at least one butyl ether spacer group.

There is also disclosed a pharmaceutically acceptable composition comprising an NK₁ antagonist or a pharmaceutically acceptable salt thereof in admixture with a polyanionic β-cyclodextrin derivative with about one to about seven sodium sulfonate groups separated from the lipophilic cavity by at least one butyl ether spacer group.

### WRITTEN DESCRIPTION OF THE INVENTION

The compositions of the present invention preferably include a Neurokinin antagonist having the following chemical structure:

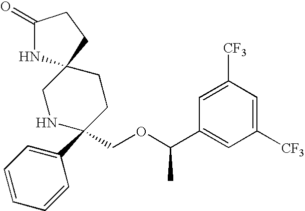

The compositions of the present invention may also include a pharmaceutically acceptable composition comprising a compound having the Formula (I): 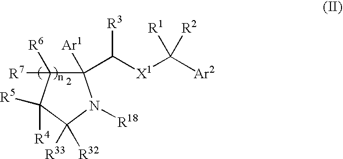
or a pharmaceutically-acceptable salt thereof, wherein
Ar¹ and Ar² are each independently selected from the group consisting of R¹⁷-heteroaryl and 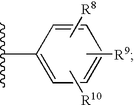
X¹ is —O—, —S—, —SO—, —SO₂—, —NR³⁴—, —N(COR¹²)— or —N(SO₂R¹⁵)—;
when X¹ is —SO—, —SO₂—, —N(COR¹²)— or —N(SO₂R¹⁵)—, then:
   R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or
when X¹ is —O—, —S— or —NR³⁴—, then:
   R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or R¹ and R², together with the carbon atom to which they are both attached, form a C═O group;
R³ is selected from the group consisting of H, C₁-C₆ alkyl, hydroxy(C₁-C₃ alkyl), C₃-C₈ cycloalkyl, —CH₂F, —CHF₂ and —CF₃;
each R⁶ is independently selected from the group consisting of H, C₁-C₆ alkyl and —OH;
each R⁷ is independently selected from the group consisting of H and C₁-C₆ alkyl;
n₂ is 1 to 4;
R⁴ and R⁵ are each independently selected from the group consisting of —(CR²⁸R²⁹)ₙ₁-G,
where,
   n₁ is 0 to 5; and
   G is H, —CF₃, —CHF₂, —CH₂F, —OH, —O—(C₁-C₆ alkyl), —OCH₂F, —OCHF₂, —OCF₃, —OCH₂CF₃, —O—(C₃-C₈ cycloalkyl), —O—(C₁-C₆)alkyl(C₃-C₈ cycloalkyl), —NR¹³R¹⁴, —SO₂NR¹³R¹⁴, —NR¹²SO₂R¹³, —NR¹²C(O)R¹⁴, —NR¹²C(O)OR¹³, —NR¹²(C(O)NR¹³R¹⁴), —C(O)NR¹³R¹⁴, —C(O)OR¹³, —C₃-C₈ cycloalkyl, (R¹⁹)ᵣ-aryl, (R¹⁹),-heteroaryl, —OC(O)R¹⁴, —OC(O)NR³R⁴₁—C(═NOR¹⁴)(R¹³)ₙ—C(O)R¹³, —C(OR¹²)(R¹³)(R¹⁴), heterocycloalkenyl optionally substituted by 1 to 4 substituents independently selected from the group consisting of R³⁰ and R³¹, 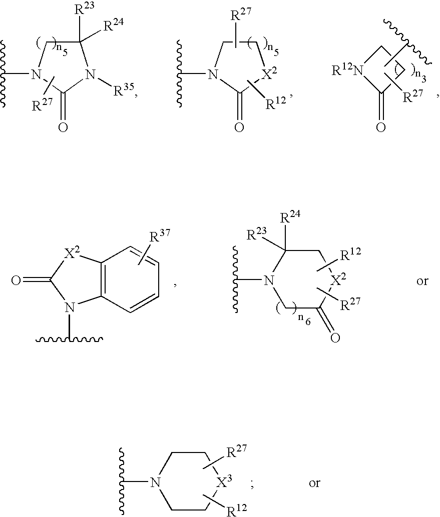
   R⁴ and R⁵ together are ═O, ═NOR¹²; or
R⁴ and R⁵, together with the carbon atom to which they are both attached, form a 4- to 8-membered heterocycloalkyl or heterocycloalkenyl ring containing 1 to 3 groups independently selected from X², provided that at least one X² is —NR³⁵—, —O—, —S—, —S(O)— or —SO₂—, the ring being optionally substituted with from 1 to 6 substituents independently selected from the group consisting of R³⁰ and R³¹;
provided that R⁴ and R⁵ are not both selected from the group consisting of H, alkyl and cycloalkyl;
further provided that, when one of R⁴ and R⁵ is —OH, then the other one of R⁴ and R⁵ is not alkyl or (R¹⁹)ᵣ-aryl;
R⁸, R⁹ and R¹⁰ are each independently selected from the group consisting of H, C₁-C₆ alkyl, C₃-C₈ cycloalkyl, —OR¹², halogen, —CN, —NO₂, —CF₃, —CHF₂, —CH₂F, —CH₂CF₃, —OCF₃, —OCHF₂, —OCH₂F, —OCH₂CF₃, —COOR¹², —CONR²¹R²², —OC(O)NR²¹R²², —OC(O)R¹², —NR² COR², —NR²¹CO₂R⁵, —NR²¹CONR²¹R²², —NR²¹SO₂R¹⁵, —NR²¹R²², —SO₂NR²¹R²², —S(O)ₙ₆R¹⁵, (R¹⁹)ᵣ-aryl and (R¹⁹)ᵣ-heteroaryl;
R¹² is H, C₁-C₆ alkyl or C₃-C₈ cycloalkyl;
R¹³ and R¹⁴ are each independently selected from the group consisting of H, C₁-C₆ alkyl, C₃-C₈ cycloalkyl, (C₃-C₈)cycloalkyl(C₁-C₆)alkyl, —CH₂CF₃, aryl and heteroaryl; or
R¹³ and R¹⁴, together with the nitrogen atom to which they are both attached, form a 4- to 7-membered saturated or unsaturated ring that is optionally substituted with —OR², where one of the carbon atoms in the ring is optionally replaced by a heteroatom selected from the group consisting of —O—, —S— and —NR³⁴—;
n₆ is 0, 1 or 2;
R¹⁵ is C₁-C₆ alkyl, C₃-C₈ cycloalkyl, —CF₃ or —CH₂CF₃;
R¹⁸ is H, C₁-C₆ alkyl, C₃-C₈ cycloalkyl, (C₃-C₈)cycloalkyl(C₁-C₆)alkyl, hydroxy(C₂-C₆)alkyl or —P(O)(OH)₂;
each R¹⁹ is a substituent on the aryl or heteroaryl ring to which it is attached, and is independently selected from the group consisting of H, C₁-C₆ alkyl, C₃-C₈ cycloalkyl, C₁-C₆ alkoxy, —OH, halogen, —CN, —NO₂, —CF₃, —CHF₂, —CH₂F, —OCF₃, —OCHF₂, —OCH₂F, —O—(C₁-C₆ alkyl), —O—(C₃-C₈ cycloalkyl), —COOR¹², —CONR²¹R²², —OC(O)NR²¹R²², —OC(O)R¹², —NR²¹R²², —NR²¹COR¹², —NR²¹COR¹², —NR²¹CONR²¹R²², —NR²¹SO₂R¹⁵ and —S(O)ₙ₆R¹⁵;
R²¹ and R²² are each independently selected from the group consisting of H, C₁-C₆ alkyl, C₃-C₈ cycloalkyl and benzyl; or
R²¹ and R²², together with the nitrogen atom to which they are both attached, form a 4- to 7-membered saturated or unsaturated ring, where one of the carbon atoms in the ring is optionally replaced by a heteroatom selected from the group consisting of —O—, —S— and —NR³⁴—;
R²³ and R²⁴ are each independently selected from the group consisting of H and C₁-C₆ alkyl; or
R²³ and R²⁴, together with the carbon atom to which they are both attached, form a C═O or cyclopropyl group;
R²⁷ is H, —OH or C₁-C₆ alkyl;
R²⁸ and R²⁹ are each independently selected from the group consisting of H and C₁-C₂ alkyl;
R³⁰ and R³¹ are each independently selected from the group consisting of H, —OH, C₁-C₆ alkyl, C₃-C₈ cycloalkyl, (C₃-C₈)cycloalkyl(C₁-C₆)alkyl and —C(O)NR¹³R¹⁴; or
R³⁰ and R³¹, together with the carbon atom to which they are both attached, form ═O, ═S, a cyclopropyl ring or ═NR³⁶;
R³² and R³³ are each independently selected from the group consisting of H and C₁-C₆ alkyl;
R³⁴ is H, C₁-C₆ alkyl, C₃-C₈ cycloalkyl, (C₃-C₈)cycloalkyl(C₁-C₆)alkyl or hydroxy(C₂-C₆)alkyl;
R³⁵ is H, C₁-C₆ alkyl, C₃-C₈ cycloalkyl, (C₃-C₈)cycloalkyl(C₁-C₆)alkyl, —P(O)(OH)₂, allyl, hydroxy(C₂-C₆)alkyl, (C₁-C₆)alkoxy(C₁-C₆)alkyl, —SO₂R¹⁵ or —(CH₂)₂—N(R¹²)—SO₂—R¹⁵;
R³⁶ is H, C₁-C₆ alkyl, C₃-C₈ cycloalkyl, (C₃-C₈)cycloalkyl(C₁-C₆)alkyl, —NO₂, —CN or OR¹²;
R³⁷ is 1 to 3 substituents independently selected from the group consisting of H, C₁-C₆ alkyl, —OH, C₁-C₆ alkoxy and halogen;
r is 1 to 3;
X² is —NR³⁵—, —O—, —S—, —S(O)—, —SO₂—, —CH₂—, —CF₂— or —CR¹²F—;
X³ is —NR³⁴—, —N(CONR¹³R¹⁴)—, —N(CO₂R¹³)—, —N(SO₂R¹⁵)—, —N(COR¹²)—, —N(SO₂NHR¹³)—, —O—, —S—, —S(O)—, —SO₂—, —CH₂—, —CF₂— or —CR¹²F—;
n₃ is 1 to 5; and
n₅ is 1 to 3;
   or a diastereomer, enantiomer, stereoisomer, regiostereomer, rotomer, tautomer, prodrug or a pharmaceutically acceptable salt thereof in admixture with a polyanionic β-cyclodextrin derivative with about one to about seven sodium sulfonate groups separated from the lipophilic cavity by at least one butyl ether spacer group.

There is also disclosed a pharmaceutically acceptable composition comprising an NK₁ antagonist or a pharmaceutically acceptable salt thereof in admixture with a polyanionic β-cyclodextrin derivative with about one to about seven sodium sulfonate salt separated from the lipophilic cavity by at least one butyl ether spacer group.

These compounds may be prepared in accordance with the procedures set forth in U.S. Patent Application Publication No. 20030158173 A1, incorporated by reference as if fully set forth herein.

The compounds of the present invention may be present in a range of from about 0.01 mg/mL to about 100 mg/mL, or about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL or about 100 mg/mL.

Solubility of Neurokinin antagonist compound, such as the compounds above, or a pharmaceutically acceptable derivative thereof, is in general low in an aqueous solution with a pH value of 5 and above. Due to the low aqueous solubility, formulation of a solution for either oral dosing, intravenous, intramuscular and subcutaneous injection is challenging.

β-Cyclodextrin sulfobutyl ether sodium salts, such as Captisol®, have been demonstrated to improve aqueous solubility of Neurokinin-1 antagonists by inclusion complexing with the compound and provides a vehicle for a solution formulation that is suitable for oral, intravenous, intramuscular or subcutaneous dosing. The ingredient may be present in an amount of from about 0.1% to about 99%, preferably 0.1% to about 40%.

Pharmaceutically acceptable salts of particular interest are salts of the (OCH₂)₄SO₃H groups, for example alkali metal salts, such as sodium salts. Preferably, the average number of O(CH₂)₄ SO₃H groups per molecule of the cyclodextrin is in the range about 1 to about 7, preferably 6.1-6.9.

More specifically, Captisol® is a sulfobutyl ether derivative of β-cyclodextrin with an average of seven sulfobutyl ether groups per cyclodextrin molecule. Because of the very low pKa of the sulfonic acid groups, Captisol® carries multiple negative charges at physiologically compatible pH values. The four-carbon butyl chain coupled with repulsion of the end group negative charges allows for an extension” of the cyclodextrin cavity. This often results in an increased possibility of inclusion complexation of the compounds with a relatively large molecular volume than has been demonstrated with other modified cyclodextrins. In addition, these derivatives impart exceptional solubility and parenteral safety to the molecule. The product is available Cydex, Inc. of Overland Park, Kans. It may reportedly be prepared in accordance with the procedures set forth in International Patent Application WO 91/11172.

Captisol® improves the solubility of the compound of Formula I in free base form, HCl salt and tosylate salt. Solubility of the above compounds was determined by equilibrating the powder with aqueous solutions containing different concentration of Captisol® at ambient temperature (20° C.).

Preferably, the formulations of the present invention are for parenteral administration, for example, intravenous or intramuscular administration. The aqueous stability of the active ingredient-cyclodextrin derivative complex may be further enhanced by lyophilisation. The cyclodextrin derivatives used in formulations according to the invention enable the finished lyophilised product to accommodate high levels of moisture without an adverse effect on stability.

Thus, to prepare an aqueous solution for intravenous injection, it is possible to use a co-solvent, e.g.-, an alcohol such as ethanol or a glycol such as polyethylene glycol or propylene glycol, or glycerin, and optionally, a hydrophilic surfactant such as Tween® 80. An oily solution injectable intramuscularly can be prepared, e.g., by solubilizing the active principle with a triglyceride or a glycerol ester. The substantially non-aqueous carrier (excipient) can be any substance that is biocompatible and liquid or soft enough body temperature. The carrier is usually hydrophobic and commonly organic, e.g., an oil or fat of vegetable, animal, mineral or synthetic origin or derivation. Preferably, but not necessarily, the carrier includes at least one chemical moiety of the kind that typifies “fatty” compounds, e.g., fatty acids, alcohols, esters, etc., i.e., a hydrocarbon chain, an ester linkage, or both. “Fatty” acids in this context include acetic, propionic and butyric acids, through straight- or branched-chain organic acids containing up to 30 or more carbon atoms. Preferably, the carrier is immiscible in water and/or soluble in the substances commonly known as fat solvents. The carrier can correspond to a reaction product of such a “fatty” compound or compounds with a hydroxy compound, e.g., a mono-hydric, di-hydric, trihydric or other polyhydric alcohol, e.g., glycerol, propanediol, lauryl alcohol, polyethylene or -propylene glycol, etc. These compounds include the fat-soluble vitamins, e.g., tocopherols and their esters, e.g., acetates sometimes produced to stabilize tocopherols. Sometimes, for economic reasons, the carrier may preferably comprise a natural, unmodified vegetable oil such as sesame oil, soybean oil, peanut oil, palm oil, or an unmodified fat. Alternatively the vegetable oil or fat may be modified by hydrogenation or other chemical means which is compatible with the present invention. The appropriate use of hydrophobic substances prepared by synthetic means is also envisioned.

Pharmaceutical compositions suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human serum albumin), toxicity agents (e.g. NaCl), preservatives (e.g. thimerosol, cresol or benylalcohol), and surfactants (e.g. tween or polysorabates) in sterile water for injection.

Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized powder in a separate compartment.

The compounds of the present invention may be administered in combination with other anti-emetics , either separately or together, such as Azasetron, Granisetron, Ondansetron, Torpisetron, DAT-582, Dolasetron, Itasetron, N-3389, Pancopride, Ramosetron, RG-12915, (R)-Zacopride, Lurosetron, E-3620, GK-128, KB-6933, KF-20170, SL-90.0539 and (−)-cis-4-amino-5-chlor-2,3-dihydro- N-[1-[3-[(3,4-dihydro4-oxo-2-pyrimidinyl)amino]-propyl]-3-methoxy-4-piperidinyl]-2,2-dimethyl-7-benzofurancarboxa mide and the pharmaceutically acceptable acid addition salts thereof. Alternatively, the composition may contain an selective serotonin reuptake inhibitor such as fluoxetine, fluvoxamine, paroxetine, sertaline, or a pharmaceutically-acceptable salt thereof. Alternatively, the composition may contain a corticosteroid such as mometasone furoate; beclomethasone dipropionate; budesonide; fluticasone; dexamethasone; flunisolide and triamcinolone.

The invention will be further described with reference to the following non- limiting examples.

### EXAMPLE 1

Captisol ® concentration (% w/v)

| | 0 | 2 | 5 | 10 |
|---|---|---|---|---|
| Solubility of | 0.05 | 3.5 | 4.7 | 10 |
| Compound I HCl salt | | | | |
| at pH 5 (pH adjusted | | | | |
| by NaOH) | | | | |
| mg/mL(free form | | | | |
| equivalent) | | | | |

### EXAMPLE 2

| | Captisol ® concentration (% w/v) | | | | |
|---|---|---|---|---|---|
| | 0 | 2 | 5 | 10 | 20 |
| Solubility of | 0.17 | 2.2 | 4.6 | 7.2 | 12 |
| Compound I free | | | | | |
| base at pH 5.2 | | | | | |
| (citric acid | | | | | |
| buffered) mg/ml | | | | | |
| (free form | | | | | |
| equivalent) | | | | | |
| Solubility of | 0.002 | 0.05 | 0.15 | 0.29 | 0.61 |
| Compound I free | | | | | |
| base at pH 7.2 | | | | | |
| (phosphate | | | | | |
| buffered) mg/ml | | | | | |
| (free form | | | | | |
| equivalent) | | | | | |

### EXAMPLE 3

Captisol ® concentration (% w/v)

| | 0 | 10 | 20 | 40 |
|---|---|---|---|---|
| Solubility of | 0.15 | 0.94 | 1.8 | 4.1 |
| Compound I Tosylate | | | | |
| salt at pH 5 (pH | | | | |
| adjusted by NaOH) | | | | |
| mg/ml (free form | | | | |
| equivalent) | | | | |

### EXAMPLE 4

Captisol ® concentration (% w/v)

| | 0 | 2 | 5 | 10 |
|---|---|---|---|---|
| Solubility of | 0.05 | 1.1 | 2.1 | 3.1 |
| Compound II HCl salt | | | | |
| at pH 5.2 (pH adjusted | | | | |
| by NaOH) mg/ml | | | | |
| (free form equivalent) | | | | |

The solutions were prepared in accordance with methods known to one of skill in the art.

The present invention has a number of benefits. Delivery systems, i.e. aqueous or mixed solvents, contain β-cyclodextrin sulfobutyl ether sodium salt, (Captisol®), or it's derivatives or a different pharmaceutically acceptable salt can be utilized for NK1 antagonist compounds set forth in the figures above and derivatives thereof to improve solubility. The concentration of Captisol® can be higher or lower than the range studied to achieve desired the desired solubility. Captisol® can be utilized in different pH's other than the pH's studied to improve solubility of the compounds having the structures set forth above. The formulations of the present invention may be used in combination with other pharmaceutical solvents or solubilization agents to improve solubility of compounds having the structures set forth above. Potentially, the formulations of the present invention may also be utilized in solution formulations to alter the in vivo oral and intramuscular or subcutaneous injection absorption profile. Additionally, the formulations of the present invention may be used in solid oral dosage forms to alter the release profile because of increased dissolution of the active agent.

The foregoing descriptions of various embodiments of the invention are representative of various aspects of the invention, and are not intended to be exhaustive or limiting to the precise forms disclosed. Many modifications and variations undoubtedly will occur to those having skill in the art. It is intended that the scope of the invention shall be fully defined solely by the appended claims.

1. A pharmaceutical composition comprising a compound having the chemical structure 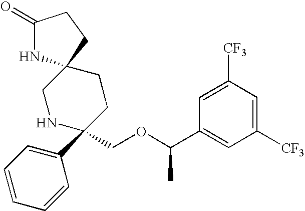
or a pharmaceutically acceptable salt thereof in admixture with a polyanionic β-cyclodextrin derivative with about one to about seven sodium sulfonate groups separated from the lipophilic cavity by at least one butyl ether spacer group.

2. The pharmaceutical composition according to claim 1, wherein the formulation has a pH of about 4 to about 8.

3. The pharmaceutical composition according to claim 1, wherein the pharmaceutically acceptable salt is a hydrochloride salt.

4. The pharmaceutical composition according to claim 1, wherein the compound is a free base.

5. The pharmaceutical composition according to claim 4, wherein the free base is buffered.

6. The pharmaceutical composition according to claim 5, wherein the free base is buffered with a citric acid or a phosphoric acid buffer.

7. A solution made by making up a lyophilized formulation, as claimed in claim 1, in water.

8. The pharmaceutical composition according to claim 1, further comprising at least one selective serotonin reuptake inhibitor.

9. The composition of claim 8, where the selective serotonin reuptake inhibitor is fluoxetine, fluvoxamine, paroxetine, sertaline, or a pharmaceutically-acceptable salt thereof.

10. The pharmaceutical composition according to claim 1, further comprising at least one serotonin 5-HT₃ receptor antagonist.

11. The composition of according to claim 10, where the serotonin 5-HT₃ receptor antagonist is selected from the group consisting of ondansetron, dolasetron, palonsetron or granisetron.

12. The pharmaceutical composition according to claim 1, further comprising a compound selected from the group consisting of a substituted benzamide or a corticosteroid.

13. The pharmaceutical composition according to claim 1, which is adapted for parenteral administration.

14. A pharmaceutically acceptable composition comprising a compound having the Formula (II): 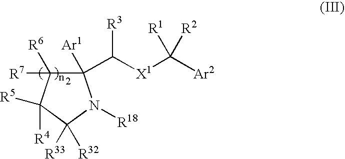
or a pharmaceutically-acceptable salt thereof, wherein
Ar¹ and Ar² are each independently selected from the group consisting of R¹⁷-heteroaryl and 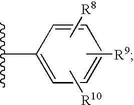
X¹ is —O—, —S—, —SO—, —SO₂—, —NR³⁴—, —N(COR¹²)— or —N(SO₂R¹⁵)—;
when X¹ is —SO—, —SO₂—, —N(COR¹²)— or —N(SO₂R¹⁵)—, then:
R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or
when X¹ is —O—, —S— or —NR³⁴—, then:
R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a C₃ to C₆ alkylene ring; or R¹ and R², together with the carbon atom to which they are both attached, form a C═O group;
R³ is selected from the group consisting of H, C₁-C₆ alkyl, hydroxy(C₁-C₃ alkyl), C₃-C₈ cycloalkyl, —CH₂F, —CHF₂ and —CF₃;
each R⁶ is independently selected from the group consisting of H, C₁-C₆ alkyl and —OH;
each R⁷ is independently selected from the group consisting of H and C₁-C₆ alkyl;
n₂ is 1 to 4;
R⁴ and R⁵ are each independently selected from the group consisting of —(CR²⁸R²⁹)ₙ₁-G,
where,
n₁ is 0 to 5; and
G is H, —CF₃, —CHF₂, —CH₂F, —OH, —O—(C₁-C₆ alkyl), —OCH₂F, —OCHF₂, —OCF₃, —OCH₂CF₃, —O—(C₃-C₈ cycloalkyl), —O—(C₁-C₆)alkyl(C₃-C₈ cycloalkyl), —NR¹³R¹⁴, —SO₂NR¹³R¹⁴, —NR¹²SO₂R¹³, —NR¹²C(O)R¹⁴, —NR¹²C(O)OR¹³, —NR¹²(C(O)NR¹³R¹⁴), —C(O)NR¹³R¹⁴, —C(O)OR¹³, —C₃-C₈ cycloalkyl, (R¹⁹)ᵣ-aryl, (R¹⁹)ᵣ-heteroaryl, —OC(O)R¹⁴, —OC(O)NR¹³R¹⁴, —C(═NOR¹⁴)(R¹³), —C(O)R¹³, —C(OR¹²)(R¹³)(R¹⁴), heterocycloalkenyl optionally substituted by 1 to 4 substituents independently selected from the group consisting of R³⁰ and R³¹, 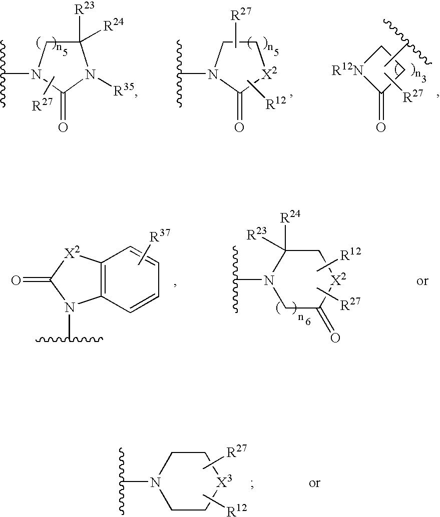
R⁴ and R⁵ together are ═O, ═NOR¹²; or
R⁴ and R⁵, together with the carbon atom to which they are both attached, form a 4- to 8-membered heterocycloalkyl or heterocycloalkenyl ring containing 1 to 3 groups independently selected from X², provided that at least one X² is —NR³⁵—, —O—, —S—, —S(O)— or —SO₂—, the ring being optionally substituted with from 1 to 6 substituents independently selected from the group consisting of R³⁰ and R³¹;
provided that R⁴ and R⁵ are not both selected from the group consisting of H, alkyl and cycloalkyl;
further provided that, when one of R⁴ and R⁵ is —OH, then the other one of R⁴ and R⁵ is not alkyl or (R¹⁹)ᵣ-aryl;
R⁸, R⁹ and R¹⁰ are each independently selected from the group consisting of H, C₁-C₆ alkyl, C₃-C₈ cycloalkyl, —OR¹², halogen, —CN, —NO₂, —CF₃, —CHF₂, —CH₂F, —CH₂CF₃, —OCF₃, —OCHF₂, —OCH₂F, —OCH₂CF₃, —COOR¹², —CONR²¹R²², —OC(O)NR²¹R²², —OC(O)R¹², —NR²¹COR¹², —NR²¹CO₂R¹⁵, —NR²¹CONR²¹R²², —NR²¹SO₂R¹⁵, —N R²¹R²², —SO₂NR²¹R²², S(O)ₙ₆R¹⁵, (R¹⁹)ᵣ-aryl and (R¹⁹)ᵣ-heteroaryl;
R¹² is H, C₁-C₆ alkyl or C₃-C₈ cycloalkyl;
R¹³ and R¹⁴ are each independently selected from the group consisting of H, C₁-C₆ alkyl, C₃-C₈ cycloalkyl, (C₃-C₈)cycloalkyl(C₁-C₆)alkyl, —CH₂CF₃, aryl and heteroaryl; or
R¹³ and R¹⁴, together with the nitrogen atom to which they are both attached, form a 4- to 7-membered saturated or unsaturated ring that is optionally substituted with —OR², where one of the carbon atoms in the ring is optionally replaced by a heteroatom selected from the group consisting of —O—, —S— and —NR³⁴—;
n₆ is 0, 1 or 2;
R¹⁵ is C₁-C₆ alkyl, C₃-C₈ cycloalkyl, —CF₃ or —CH₂CF₃;
R¹⁸ is H, C₁-C₆ alkyl, C₃-C₈ cycloalkyl, (C₃-C₈)cycloalkyl(C₁-C₆)alkyl, hydroxy(C₂-C₆)alkyl or —P(O)(OH)₂;
each R¹⁹ is a substituent on the aryl or heteroaryl ring to which it is attached, and is independently selected from the group consisting of H, C₁-C₆ alkyl, C₃-C₈ cycloalkyl, C₁-C₆ alkoxy, —OH, halogen, —CN, —NO₂, —CF₃, —CHF₂, —CH₂F, —OCF₃, —OCHF₂, —OCH₂F, —O—(C₁-C₆ alkyl), —O—(C₃-C₈ cycloalkyl), —COOR¹², —CONR²¹R²², —OC(O)NR²¹R²², —OC(O)R¹², —NR²¹R²², —NR²¹COR¹², —NR²¹CO₂R¹², —NR²¹CONR²¹ R²², —NR²¹SO₂R¹⁵ and —S(O)ₙ₆R¹⁵;
R²¹ and R²² are each independently selected from the group consisting of H, C₁-C₆ alkyl, C₃-C₈ cycloalkyl and benzyl; or
R²¹ and R²², together with the nitrogen atom to which they are both attached, form a 4- to 7-membered saturated or unsaturated ring, where one of the carbon atoms in the ring is optionally replaced by a heteroatom selected from the group consisting of —O—, —S— and —NR³⁴—;
R²³ and R²⁴ are each independently selected from the group consisting of H and C₁-C₆ alkyl; or
R²³ and R²⁴, together with the carbon atom to which they are both attached, form a C═O or cyclopropyl group;
R²⁷ is H, —OH or C₁-C₆ alkyl;
R²⁸ and R²⁹ are each independently selected from the group consisting of H and C₁-C₂ alkyl;
R³⁰ and R³¹ are each independently selected from the group consisting of H, —OH, C₁-C₆ alkyl, C₃-C₈ cycloalkyl, (C₃-C₈)cycloalkyl(C₁-C₆)alkyl and —C(O)NR¹³R¹⁴; or
R³⁰ and R³¹, together with the carbon atom to which they are both attached, form ═O, ═S, a cyclopropyl ring or ═NR³⁶;
R³² and R³³ are each independently selected from the group consisting of H and C₁-C₆ alkyl;
R³⁴ is H, C₁-C₆ alkyl, C₃-C₈ cycloalkyl, (C₃-C₈)cycloalkyl(C₁-C₆)alkyl or hydroxy(C₂-C₆)alkyl;
R³⁵ is H, C₁-C₆ alkyl, C₃-C₈ cycloalkyl, (C₃-C₈)cycloalkyl(C₁-C₆)alkyl, —P(O)(OH)₂, allyl, hydroxy(C₂-C₆)alkyl, (C₁-C₆)alkoxy(C₁-C₆)alkyl, —SO₂R¹⁵ or —(CH₂)₂—N(R¹²)—SO₂—R¹⁵;
R³⁶ is H, C₁-C₆ alkyl, C₃-C₈ cycloalkyl, (C₃-C₈)cycloalkyl(C₁-C₆)alkyl, —NO₂, —CN or OR¹²;
R³⁷ is 1 to 3 substituents independently selected from the group consisting of H, C₁-C₆ alkyl, —OH, C₁-C₆ alkoxy and halogen;
r is 1 to 3;
X² is —NR³⁵—, —O—, —S—, —S(O)—, —SO₂—, —CH₂—, —CF₂— or —CR¹²F—;
X³ is —NR³⁴—, —N(CONR¹³R¹⁴)—, —N(CO₂R¹³)—, —N(SO₂R¹⁵)—, —N(COR¹²)—, —N(SO₂NHR¹³)—, —O—, —S—, —S(O)—, —SO₂—, —CH₂—, —CF₂— or —CR¹²F—;
n₃ is 1 to 5; and
n₅ is 1 to 3;
or a diastereomer, enantiomer, stereoisomer, regiostereomer, rotomer, tautomer or prodrug thereof a pharmaceutically acceptable salt thereof in admixture with a polyanionic β-cyclodextrin derivative with about one to about 7 sodium sulfonate groups separated from the lipophilic cavity by at least one butyl ether spacer group.

15. The pharmaceutical composition according to claim 14, wherein the formulation has a pH of about 4 to about 8.

16. The pharmaceutical composition according to claim 14, wherein the compound is a free base.

17. The pharmaceutical composition according to claim 4, wherein the free base is buffered.

18. A solution made by making up a lyophilized formulation, as claimed in claim 14, in water.

19. The pharmaceutical composition according to claim 14, further comprising at least one compound selected from the group consisting of selective serotonin reuptake inhibitors, serotonin 5-HT₃ receptor antagonist, a substituted benzamide or a corticosteroid.

20. The pharmaceutical composition according to claim 14, which is adapted for parenteral administration.

21. A pharmaceutically acceptable composition comprising an NK₁ antagonist or a pharmaceutically acceptable salt thereof in admixture with a polyanionic β-cyclodextrin derivative with about one to about seven sodium sulfonate groups separated from the lipophilic cavity by at least one butyl ether spacer group.
